(19) United States
(12) Patent Application Publication
(10) Pub. No.: US 2014/0113874 A1
(43) Pub. Date: Apr. 24, 2014

(54) Modified Compstatin With Improved Stability And Binding Properties

(71) Applicants: John D. Lambris, Philadelphia, PA (US); Wilfred A. Van Der Donk, Champaign, IL (US)

(72) Inventors: John D. Lambris, Philadelphia, PA (US); Wilfred A. Van Der Donk, Champaign, IL (US)

(73) Assignee: THE TRUSTEES OF THE UNIVERSITY OF PENNSYLVANIA, Philadelphia, PA (US)

(21) Appl. No.: 13/825,775

(22) PCT Filed: Sep. 21, 2011
(86) PCT No.: PCT/US11/52442

§ 371 Date: Dec. 23, 2013

Related U.S. Application Data
(60) Provisional application No. 61/385,711, filed on Sep. 23, 2010.

(51) Int. Cl. A61K 38/10; C07K 7/02

(52) U.S. Cl. CPC A61K 38/10; C07K 7/02
USPC 514/21.4; 530/323; 514/21.5; 530/327

(57) ABSTRACT

Compounds comprising peptides capable of binding C3 protein and inhibiting complement activation are disclosed. These cyclic compounds are modified to improve stability while maintaining substantially equivalent complement activation-inhibitory activity as compared with currently available compounds. The compounds comprise compstatin analogs in which the disulfide bond between C2 and C12 is modified via a thioether bond to form a cystathionine.

### GOVERNMENT SUPPORT

Pursuant to 35 U.S.C. §202(c), it is acknowledged that the United States government may have certain rights in the invention described herein, which was made in part with funds from the National Institutes of Health under Grant No. GM 62134.

### FIELD OF THE INVENTION

This invention relates to activation of the complement cascade in the body. In particular, this invention provides peptides and peptidomimetics capable of binding the C3 protein and inhibiting complement activation.

### BACKGROUND OF THE INVENTION

Various publications, including patents, published applications, technical articles and scholarly articles are cited throughout the specification. Each of these cited publications is incorporated by reference herein, in its entirety.

The human complement system is a powerful player in the defense against pathogenic organisms and the mediation of immune responses. Complement can be activated through three different pathways: the classical, lectin, and alternative pathways. The major activation event that is shared by all three pathways is the proteolytic cleavage of the central protein of the complement system, C3, into its activation products C3a and C3b by C3 convertases. Generation of these fragments leads to the opsonization of pathogenic cells by C3b and iC3b, a process that renders them susceptible to phagocytosis or clearance, and to the activation of immune cells through an interaction with complement receptors. Deposition of C3b on target cells also induces the formation of new convertase complexes and thereby initiates a self-amplification loop.

An ensemble of plasma and cell surface-bound proteins carefully regulates complement activation to prevent host cells from self-attack by the complement cascade. However, excessive activation or inappropriate regulation of complement can lead to a number of pathologic conditions, ranging from autoimmune to inflammatory diseases. The development of therapeutic complement inhibitors is therefore highly desirable. In this context, C3 and C3b have emerged as promising targets because their central role in the cascade allows for the simultaneous inhibition of the initiation, amplification, and downstream activation of complement.

Compstatin was the first non-host-derived complement inhibitor that was shown to be capable of blocking all three activation pathways (Sahu et al., 1996, J Immunol 157: 884-91; U.S. Pat. No. 6,319,897). This cyclic tridecapeptide binds to both C3 and C3b and prevents the cleavage of native C3 by the C3 convertases. Its high inhibitory efficacy was confirmed by a series of studies using experimental models that pointed to its potential as a therapeutic agent (Fiane et al., 1999a, Xenotransplantation 6: 52-65; Fiane et al., 1999b, Transplant Proc 31:934-935; Nilsson et al., 1998 Blood 92: 1661-1667; Ricklin & Lambris, 2008, Adv Exp Med Biol 632: 273-292; Schmidt et al., 2003, J Biomed Mater Res A 66: 491-499; Soulika et al., 2000, Clin Immunol 96: 212-221). Progressive optimization of compstatin has yielded analogs with improved activity (Ricklin & Lambris, 2008, supra; WO2004/026328; WO2007/062249). One of these analogs is currently being tested in clinical trials for the treatment of age-related macular degeneration (AMD), the leading cause of blindness in elderly patients in industrialized nations (Coleman et al., 2008, Lancet 372: 1835-1845; Ricklin & Lambris, 2008, supra). In view of its therapeutic potential in AMD and other diseases, further optimization of compstatin to achieve an even greater efficacy is of considerable importance.

Earlier structure-activity studies have identified the cyclic nature of the compstatin peptide and the presence of both a β-turn and hydrophobic cluster as key features of the molecule (Morikis et al., 1998, Protein Sci 7: 619-627; WO99/13899; Morikis et al., 2002, J Biol Chem 277:14942-14953; Ricklin & Lambris, 2008, supra). Hydrophobic residues at positions 4 and 7 were found to be of particular importance, and their modification with unnatural amino acids generated an analog with 264-fold improved activity over the original compstatin peptide (Katragadda et al., 2006, J Med Chem 49: 4616-4622; WO2007/062249).

While previous optimization steps have been based on combinatorial screening studies, solution structures, and computational models (Chiu et al., 2008, Chem Biol Drug Des 72: 249-256; Mulakala et al., 2007, Bioorg Med Chem 15: 1638-1644; Ricklin & Lambris, 2008, supra), the recent publication of a co-crystal structure of compstatin complexed with the complement fragment C3c (Janssen et al., 2007, J Biol Chem 282: 29241-29247; WO2008/153963) represents an important milestone for initiating rational optimization. The crystal structure revealed a shallow binding site at the interface of macroglobulin (MG) domains 4 and 5 of C3c and showed that 9 of the 13 amino acids were directly involved in the binding, either through hydrogen bonds or hydrophobic effects. As compared to the structure of the compstatin peptide in solution (Morikis et at, 1998, supra), the bound form of compstatin experienced a conformational change, with a shift in the location of the β-turn from residues 5-8 to 8-11 (Janssen et al., 2007, supra; WO2008/153963).

In view of the foregoing, it is clear that the development of modified compstatin peptides or mimetics with even greater activity would constitute a significant advance in the art.

### SUMMARY OF THE INVENTION

The present invention provides analogs of the complement-inhibiting peptide, compstatin, ICVVQDWGHHRCT (disulfide C2-C12); SEQ ID NO:1), which maintain improved complement-inhibiting activity as compared to compstatin, and which also possess improved stability characteristics.

One aspect of the invention features a compound comprising a modified compstatin peptide (ICVVQDWGHHRCT (cyclic C2-C12); SEQ ID NO:1) or analog thereof, in which the disulfide bond between C2 and C12 is replaced with a thioether bond. In one embodiment, a cystathionine is formed. The cystathionine can be delta-cystathionine or a gamma-cystathionine.

The aforementioned compound can further comprise one or more of the following modifications: (1) replacement of His at position 9 with Ala; (2) replacement of Val at position 4 with Trp or an analog of Trp; (3) replacement of Trp at position 7 with an analog of Trp; (4) acetylation of the N-terminal residue; (5) modification of Gly at position 8 to constrain the backbone conformation at that location; and (6) replacing the Thr at position 13 with Ile, Leu, Nle, N-methyl Thr or N-methyl Ile. In a particular embodiment, the analog of Trp at position 4 is 1-methyl Trp or 1-formyl Trp. In another embodiment, the analog of Trp at position 7 is a halogenated Trp. In another embodiment, the backbone is constrained by replacing the Gly at position 8 (Gly8) with Nα-methyl Gly.

In another embodiment, the compound is a compstatin analog comprising a peptide having a sequence of SEQ ID NO:2, which is:

Xaa1-Cys-Val-Xaa2-Gln-Asp-Xaa3-Gly-Xaa4-His-Arg-
Cys-Xaa5

(cystathionine C2-C12, wherein one of C2 or C2 is modified to homocysteine) in which Gly at position 8 is modified to constrain the backbone conformation at that location; wherein: Xaa1 is Ile, Val, Leu, Ac-Ile, Ac-Val, Ac-Leu or a dipeptide comprising Gly-Ile; Xaa2 is Trp or an analog of Trp, wherein the analog of Trp has increased hydrophobic character as compared with Trp; Xaa3 is Trp or an analog of Trp comprising a chemical modification to its indole ring wherein the chemical modification increases the hydrogen bond potential of the indole ring; Xaa4 is His, Ala, Phe or Trp; and Xaa5 is Thr, Ile, Leu, Nle, N-methyl Thr or N-methyl Ile, wherein a carboxy terminal —OH of any of the Thr, Ile, Leu, Nle, N-methyl Thr or N-methyl Ile optionally is replaced by —NH₂. More particularly, compounds of this embodiment have the following modifications: the cystathionine is a delta-cystathionine; the Gly at position 8 is N-methylated; Xaa1 is Ac-Ile; Xaa2 is Trp, 1-methyl-Trp or 1-formyl-Trp; Xaa3 is Trp; Xaa4 is Ala; and Xaa5 is Thr, Ile, Leu, Nle, N-methyl Thr or N-methyl Ile, and most particularly Ile, N-methyl Thr or N-methyl Ile. An exemplary compound comprises one of SEQ ID NOS: 5 or 7.

In some embodiments, the compound comprises a peptide produced by expression of a polynucleotide encoding the peptide. In other embodiments, the compound is produced at least in part by peptide synthesis. A combination of synthetic methods can also be used.

Another aspect of the invention features a compound of any of the preceding claims, further comprising an additional component that extends the in vivo retention of the compound. The additional component is polyethylene glycol (PEG) in one embodiment. The additional component is an albumin binding small molecule in another embodiment. In another embodiment, the additional component is an albumin binding peptide. The albumin binding peptide may comprise the sequence RLIEDICLPRWGCLWEDD (SEQ ID NO: 8). Optionally, the compound and the albumin binding peptide are separated by a spacer. The spacer can be a polyethylene glycol (PEG) molecule, such as mini-PEG or mini-PEG 3.

The compstatin analogs and conjugates of the invention are of practical utility for any purpose for which compstatin itself is utilized, as known in the art and described in greater detail herein. Certain of these uses involve the formulation of the compounds into pharmaceutical compositions for administration to a patient. Such formulations may comprise pharmaceutically acceptable salts of the compounds, as well as one or more pharmaceutically acceptable diluents, carriers excipients, and the like, as would be within the purview of the skilled artisan.

Another aspect of the invention features compound that inhibits complement activation, comprising a non-peptide or partial peptide mimetic of SEQ ID NO:5 or SEQ ID NO:7, wherein the compound binds C3 and inhibits complement activation with at least 500-fold greater activity than does a peptide comprising SEQ ID NO:1 under equivalent assay conditions.

Other features and advantages of the present invention will be understood by reference to the detailed description, drawings and examples that follow.

### BRIEF DESCRIPTION OF THE DRAWINGS

FIG. 1. Sequences of thioether compstatin analogs and schematic representation of the orientation of their thioether bonds. A—4W9A gamma-Cth (γ-cystathionine, SEQ ID NO:4); B—4W9A delta-Cth (δ-cystathionine, SEQ ID NO:5); C—CP20 (δ-cystathionine; SEQ ID NO: 7).

FIG. 2. Kinetic ranking of compstatin analogs. 1 uM of peptide was injected over captured C3b at a surface density of 3000 RU. The signals are overlaid to show relative differences in their association and dissociation phases. A schematic representation of the assay can be seen on the right.

FIG. 3. Single-cycle kinetics analysis of the cystathionine compstatin analogs and corresponding disulfide bond controls. Increasing concentrations were injected over a C3b surface of 3000 RU density one after another in a single cycle. The responses were corrected for non-specific binding by subtraction of the response of the untreated, reference surface and blank injections. The processed signals were fitted to a 1:1 binding model (dotted simulation curves) and kinetic constants kₐ and k_{d} were extracted.

FIG. 4. Representative result of inhibition of antigen-antibody complex—initiated complement activation by compstatin analogs. The percentage of inhibition is plotted against the peptide concentration and compared to 4W9A and CP20. The relative activity is CP20>delta-Cth CP20>4W9A>delta-Cth>gamma-Cth. A schematic representation of the assay can be seen on the right.

### DETAILED DESCRIPTION OF ILLUSTRATIVE EMBODIMENTS

### Definitions

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

The term “about” as used herein when referring to a measurable value such as an amount, a temporal duration, and the like, is meant to encompass variations of ±20% or ±10%, in some embodiments ±5%, in some embodiments ±1%, and in some embodiments ±0.1% from the specified value, as such variations are appropriate to make and used the disclosed compounds and compositions.

The term “compstatin” as used herein refers to a peptide comprising SEQ ID NO:1, ICVVQDWGHHRCT (cyclic C2-C12 by way of a disulfide bond). The term “compstatin analog” refers to a modified compstatin comprising substitutions of natural and unnatural amino acids, or amino acid analogs, as well as modifications within or between various amino acids, as described in greater detail herein, and as known in the art. When referring to the location particular amino acids or analogs within compstatin or compstatin analogs, those locations are sometimes referred to as “positions” within the peptide, with the positions numbered from 1 (Ile in compstatin) to 13 (Thr in compstatin). For example, the Gly residue occupies “position 8.”

The terms “pharmaceutically active” and “biologically active” refer to the ability of the compounds of the invention to bind C3 or fragments thereof and inhibit complement activation. This biological activity may be measured by one or more of several art-recognized assays, as described in greater detail herein.

As used herein, “alkyl” refers to an optionally substituted saturated straight, branched, or cyclic hydrocarbon having from about 1 to about 10 carbon atoms (and all combinations and subcombinations of ranges and specific numbers of carbon atoms therein), with from about 1 to about 7 carbon atoms being preferred. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2,2-dimethylbutyl, and 2,3-dimethylbutyl. The term “lower alkyl” refers to an optionally substituted saturated straight, branched, or cyclic hydrocarbon having from about 1 to about 5 carbon atoms (and all combinations and subcombinations of ranges and specific numbers of carbon atoms therein). Lower alkyl groups include, but are not limited to, methyl, ethyl, n-propyl, isopropyl, n-butyl, isobutyl, t-butyl, n-pentyl, cyclopentyl, isopentyl and neopentyl.

As used herein, “halo” refers to F, Cl, Br or I.

As used herein, “alkanoyl”, which may be used interchangeably with “acyl”, refers to an optionally substituted straight or branched aliphatic acylic residue having from about 1 to about 10 carbon atoms (and all combinations and subcombinations of ranges and specific numbers of carbon atoms therein), with from about 1 to about 7 carbon atoms being preferred. Alkanoyl groups include, but are not limited to, formyl, acetyl, propionyl, butyryl, isobutyryl pentanoyl, isopentanoyl, 2-methyl-butyryl, 2,2-dimethylpropionyl, hexanoyl, heptanoyl, octanoyl, and the like. The term “lower alkanoyl” refers to an optionally substituted straight or branched aliphatic acylic residue having from about 1 to about 5 carbon atoms (and all combinations and subcombinations of ranges and specific numbers of carbon atoms therein. Lower alkanoyl groups include, but are not limited to, formyl, acetyl, n-propionyl, iso-propionyl, butyryl, iso-butyryl, pentanoyl, iso-pentanoyl, and the like.

As used herein, “aryl” refers to an optionally substituted, mono- or bicyclic aromatic ring system having from about 5 to about 14 carbon atoms (and all combinations and subcombinations of ranges and specific numbers of carbon atoms therein), with from about 6 to about 10 carbons being preferred. Non-limiting examples include, for example, phenyl and naphthyl.

As used herein, “aralkyl” refers to alkyl as defined above, bearing an aryl substituent and having from about 6 to about 20 carbon atoms (and all combinations and subcombinations of ranges and specific numbers of carbon atoms therein), with from about 6 to about 12 carbon atoms being preferred. Aralkyl groups can be optionally substituted. Non-limiting examples include, for example, benzyl, naphthylmethyl, diphenylmethyl, triphenylmethyl, phenylethyl, and diphenylethyl.

As used herein, the terms “alkoxy” and “alkoxyl” refer to an optionally substituted alkyl-O— group wherein alkyl is as previously defined. Exemplary alkoxy and alkoxyl groups include methoxy, ethoxy, n-propoxy, i-propoxy, n-butoxy, and heptoxy, among others.

As used herein, “carboxy” refers to a —C(C═O)OH group.

As used herein, “alkoxycarbonyl” refers to a —C(C═O)O-alkyl group, where alkyl is as previously defined.

As used herein, “aroyl” refers to a —C(C═O)-aryl group, wherein aryl is as previously defined. Exemplary aroyl groups include benzoyl and naphthoyl.

Typically, substituted chemical moieties include one or more substituents that replace hydrogen at selected locations on a molecule. Exemplary substituents include, for example, halo, alkyl, cycloalkyl, aralkyl, aryl, sulfhydryl, hydroxyl (—OH), alkoxyl, cyano (—CN), carboxyl (—COOH), acyl (alkanoyl: —C(═O)R); —C(═O)O-alkyl, aminocarbonyl (—C(C═O)NH₂), —N-substituted aminocarbonyl (—C(═O)NHR″), CF₃, CF₂CF₃, and the like. In relation to the aforementioned substituents, each moiety R″ can be, independently, any of H, alkyl, cycloalkyl, aryl, or aralkyl, for example.

As used herein, “L-amino acid” refers to any of the naturally occurring levorotatory alpha-amino acids normally present in proteins or the alkyl esters of those alpha-amino acids. The term D-amino acid” refers to dextrorotatory alpha-amino acids. Unless specified otherwise, all amino acids referred to herein are L-amino acids.

“Hydrophobic” or “nonpolar” are used synonymously herein, and refer to any inter- or intra-molecular interaction not characterized by a dipole.

“PEGylation” refers to the reaction in which at least one polyethylene glycol (PEG) moiety, regardless of size, is chemically attached to a protein or peptide to form a PEG-peptide conjugate. “PEGylated means that at least one PEG moiety, regardless of size, is chemically attached to a peptide or protein. The term PEG is generally accompanied by a numeric suffix that indicates the approximate average molecular weight of the PEG polymers; for example, PEG-8,000 refers to polyethylene glycol having an average molecular weight of about 8,000.

As used herein, “pharmaceutically-acceptable salts” refers to derivatives of the disclosed compounds wherein the parent compound is modified by making acid or base salts thereof. Examples of pharmaceutically-acceptable salts include, but are not limited to, mineral or organic acid salts of basic residues such as amines; alkali or organic salts of acidic residues such as carboxylic acids; and the like. Thus, the term “acid addition salt” refers to the corresponding salt derivative of a parent compound that has been prepared by the addition of an acid. The pharmaceutically-acceptable salts include the conventional salts or the quaternary ammonium salts of the parent compound formed, for example, from inorganic or organic acids. For example, such conventional salts include, but are not limited to, those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Certain acidic or basic compounds of the present invention may exist as zwitterions. All forms of the compounds, including free acid, free base, and zwitterions, are contemplated to be within the scope of the present invention.

### DESCRIPTION

In accordance with the present invention, analogs of the complement-inhibiting peptide, compstatin, ICVVQDWGHHRCT (disulfide C2-C12; SEQ ID NO:1) are provided, in which improved complement-inhibiting activity as compared to compstatin is maintained, and which also possess improved stability characteristics.

Compstatin analogs synthesized in accordance with previous approaches have been shown to possess improved activity as compared with the parent peptide, i.e., up to about 99-fold (Mallik, B. et al, 2005, supra; WO2004/026328), and up to about 264-fold (Katragadda et al., 2006, supra; WO2007/062249), and further up to over 500-fold (WO2010/127336). The analogs produced in accordance with the present invention demonstrate activity that is substantially the same as that of certain of the aforementioned analogs, and also possess equivalent or improved stability characteristics, via modification of the C2-C12 disulfide bond to a thioether bond, to form a cystathionine.

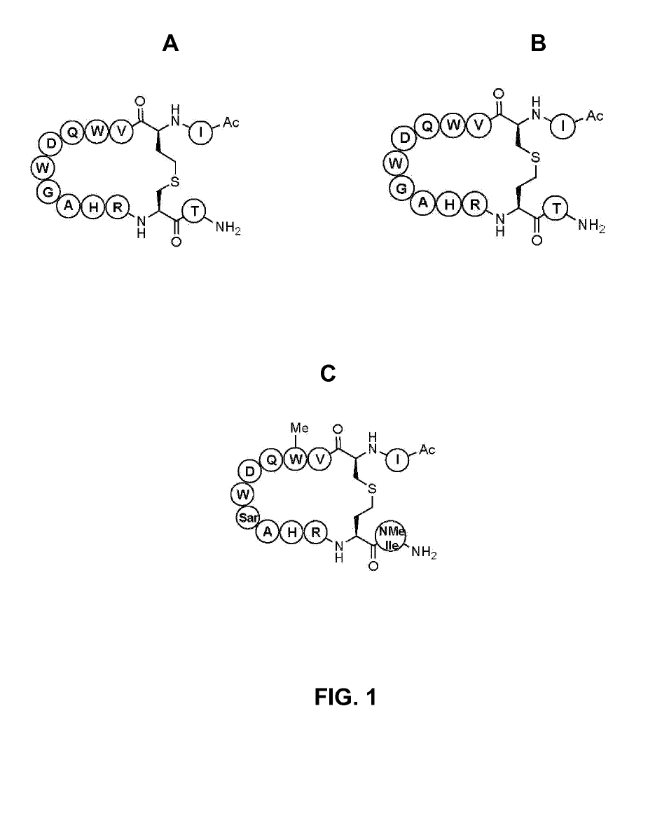

The table below shows amino acid sequence and complement inhibitory activities of selected exemplary analogs with thioether modifications, as compared with their counterpart analogs having a C2-C12 disulfide bond. Certain of the thioether analogs are also shown diagrammatically in FIG. 1 as well. The selected analogs are referred to by specific modifications of designated positions (1-13) as compared to compstatin.

### Comparison of Disulfide Bonded and Delta-Cystathionine Compstatin Analogs

| SEQ ID | | C2-C12 | | | |
|---|---|---|---|---|---|
| NO: | Nickname | bond type | Sequence | IC₅₀ (nM) | K_{D} (nM) |
| 3 | 4W9A | disulfide | Ac-Ic[CVWQDWGAHRC]T-NH₂ | 1.9 ± 1.3 | 150 ± 19 |
| 5 | 4W9A | thioether | Ac-Ic[CVWQDWGAHRC]T-NH₂ | 3.3 ± 2 | 236 ± 20 |
| | delta-Cth | | | | |
| 6 | CP20 | disulfide | AC-Ic[CV(^{1-Me}W)QDW(^{N-Me}G)AHRC](^{N-Me}I)-NH₂ | 0.13 ± 0.05 | 2.4 ± 0.6 |
| 7 | CP20 | thioether | Ac-Ic[CV(^{1-Me}W)QDW(^{N-Me}G)AHRC](^{N-Me}I)-NH₂ | 0.26 ± 0.09 | 9 ± 1.8 |
| | delta-Cth | | | | |

One modification in accordance with the present invention comprises replacement of the C2-C12 disulfide bond with addition of a CH₂ to form a homocysteine at C2 or C12, and introduction of a thioether bond, to form a cystathionine. In one embodiment, the cystathionine is a gamma-cystathionine. In another embodiment, the cystathionine is a delta-cystathionine. Another modification in accordance with the present invention comprises replacement of the C2-C12 disulfide bond with a thioether bond without the addition of a CH2, thereby forming a lantithionine.

Without intending to be bound or limited by theory, it is noted that replacement of a disulfide bond with a thioether bond in a peptide or protein has been shown to increase the in vivo stability of the peptide or protein, possibly by rendering it less susceptible to proteolytic degradation, among other possible effects. However, such modifications can have a detrimental effect on the biological activity of the protein. The inventors have demonstrated in accordance with the present invention that the type and position of the thioether bond in the compstatin analog can affect the binding affinity and complement inhibitory activity of the peptides. Thus, for instance, as described in greater detail in Example 1, modification of compstatin to form a delta-cystathionine yields analogs with activity that is substantially the same as their disulfide-bonded counterparts.

The above-described modifications of the C2-C12 bond can be combined with other modifications of compstatin previously shown to improve activity, to produce peptides with significantly improved complement inhibiting activity. For example, acetylation of the N-terminus typically increases the complement-inhibiting activity of compstatin and its analogs. Accordingly, addition of an acyl group at the amino terminus of the peptide, including but not limited to N-acetylation, is one preferred embodiment of the invention, of particular utility when the peptides are prepared synthetically. However, it is sometimes of advantage to prepare the peptides by expression of a peptide-encoding nucleic acid molecule in a prokaryotic or eukaryotic expression system, or by in vitro transcription and translation. For these embodiments, the naturally-occurring N-terminus may be utilized.

As another example, it is known that substitution of Ala for His at position 9 improves activity of compstatin and is a preferred modification of the peptides of the present invention as well. It has also been determined that substitution of Tyr for Val at position 4 can result in a modest improvement in activity (Klepeis et al., 2003, J Am Chem Soc 125: 8422-8423).

It was disclosed in WO2004/026328 and WO2007/0622249 that Trp and certain Trp analogs at position 4, as well as certain Trp analogs at position 7, especially combined with Ala at position 9, yields many-fold greater activity than that of compstatin. These modifications are used to advantage in the present invention as well.

In particular, peptides comprising 5-fluoro-l-tryptophan or either 5-methoxy-, 5-methyl- or 1-methyl-tryptophan, or 1-formyl-tryptophan at position 4 have been shown to possess 31-264-fold greater activity than does compstatin. Particularly preferred are 1-methyl and 1-formyl tryptophan. It is believed that an indole ‘N’-mediated hydrogen bond is not necessary at position 4 for the binding and activity of compstatin. The absence of this hydrogen bond or reduction of the polar character by replacing hydrogen with lower alkyl, alkanoyl or indole nitrogen at position 4 enhances the binding and activity of compstatin. Without intending to be limited to any particular theory or mechanism of action, it is believed that a hydrophobic interaction or effect at position 4 strengthens the interaction of compstatin with C3. Accordingly, modifications of Trp at position 4 (e.g., altering the structure of the side chain according to methods well known in the art), or substitutions at position 4 or position 7 of Trp analogs that maintain or enhance the aforementioned hydrophobic interaction are contemplated in the present invention as an advantageous modification in combination with the modifications at positions 8 and 13 as described above. Such analogs are well known in the art and include, but are not limited to the analogs exemplified herein, as well as unsubstituted or alternatively substituted derivatives thereof. Examples of suitable analogs may be found by reference to the following publications, and many others: Beene, et al., 2002, Biochemistry 41: 10262-10269 (describing, inter alia, singly- and multiply-halogenated Trp analogs); Babitzky & Yanofsky, 1995, J. Biol. Chem. 270: 12452-12456 (describing, inter alia, methylated and halogenated Trp and other Trp and indole analogs); and U.S. Pat. Nos. 6,214,790, 6,169,057, 5,776,970, 4,870,097, 4,576,750 and 4,299,838. Trp analogs may be introduced into the compstatin peptide by in vitro or in vivo expression, or by peptide synthesis, as known in the art.

In certain embodiments, Trp at position 4 of compstatin is replaced with an analog comprising a 1-alkyl substituent, more particularly a lower alkyl (e.g., C₁-C₅) substituent as defined above. These include, but are not limited to, N(α) methyl tryptophan, N(α) formyl tryptophan and 5-methyltryptophan. In other embodiments, Trp at position 4 of compstatin is replaced with an analog comprising a 1-alkanoyl substituent, more particularly a lower alkanoyl (e.g., C₁-C₅) substituent as defined above. In addition to exemplified analogs, these include but are not limited to 1-acetyl-L-tryptophan and L-β-homotryptophan.

It was disclosed in WO2007/0622249 that incorporation of 5-fluoro-l-tryptophan at position 7 in compstatin increased enthalpy of the interaction between compstatin and C3, relative to wildtype compstatin, whereas incorporation of 5-fluoro-tryptophan at position 4 in compstatin decreased the enthalpy of this interaction. Accordingly, modifications of Trp at position 7, as described in WO02007/0622249, are contemplated as useful modifications in combination with the modifications to positions 8 and 13 as described above.

Other modifications are described in WO2010/127336. One modification disclosed in that document comprises constraint of the peptide backbone at position 8 of the peptide. In a particular embodiment, the backbone is constrained by replacing glycine at position 8 (Gly⁸) with N-methyl glycine. Another modification disclosed in that document comprises replacing Thr at position 13 with Ile, Leu, Nle (norleucine), N-methyl Thr or N-methyl Ile.

The modified compstatin peptides of the present invention may be prepared by various synthetic methods of peptide synthesis via condensation of one or more amino acid residues, in accordance with conventional peptide synthesis methods. For example, peptides are synthesized according to standard solid-phase methodologies, such as may be performed on an Applied Biosystems Model 431A peptide synthesizer (Applied Biosystems, Foster City, Calif.), according to manufacturer's instructions. Other methods of synthesizing peptides or peptidomimetics, either by solid phase methodologies or in liquid phase, are well known to those skilled in the art. During the course of peptide synthesis, branched chain amino and carboxyl groups may be protected/deprotected as needed, using commonly-known protecting groups. Modification utilizing alternative protecting groups for peptides and peptide derivatives will be apparent to those of skill in the art.

Alternatively, certain peptides of the invention may be produced by expression in a suitable prokaryotic or eukaryotic system. For example, a DNA construct may be inserted into a plasmid vector adapted for expression in a bacterial cell (such as E. coli) or a yeast cell (such as Saccharomyces cerevisiae), or into a baculovirus vector for expression in an insect cell or a viral vector for expression in a mammalian cell. Such vectors comprise the regulatory elements necessary for expression of the DNA in the host cell, positioned in such a manner as to permit expression of the DNA in the host cell. Such regulatory elements required for expression include promoter sequences, transcription initiation sequences and, optionally, enhancer sequences.

The peptides can also be produced by expression of a nucleic acid molecule in vitro or in vivo. A DNA construct encoding a concatemer of the peptides, the upper limit of the concatemer being dependent on the expression system utilized, may be introduced into an in vivo expression system. After the concatemer is produced, cleavage between the C-terminal Asn and the following N-terminal G is accomplished by exposure of the polypeptide to hydrazine.

The peptides produced by gene expression in a recombinant procaryotic or eucaryotic system may be purified according to methods known in the art. A combination of gene expression and synthetic methods may also be utilized to produce compstatin analogs. For example, an analog can be produced by gene expression and thereafter subjected to one or more post-translational synthetic processes, e.g., to modify the N- or C- terminus or to cyclize the molecule.

Advantageously, peptides that incorporate unnatural amino acids, e.g., methylated amino acids, may be produced by in vivo expression in a suitable prokaryotic or eukaryotic system. For example, methods such as those described by Katragadda & Lambris (2006, Protein Expression and Purification 47: 289-295) to introduce unnatural Trp analogs into compstatin via expression in E. coli auxotrophs may be utilized to introduce N-methylated or other unnatural amino acids at selected positions of compstatin.

The structure of compstatin is known in the art, and the structures of the foregoing analogs are determined by similar means. Once a particular desired conformation of a short peptide has been ascertained, methods for designing a peptide or peptidomimetic to fit that conformation are well known in the art. Of particular relevance to the present invention, the design of peptide analogs may be further refined by considering the contribution of various side chains of amino acid residues, as discussed above (i.e., for the effect of functional groups or for steric considerations).

It will be appreciated by those of skill in the art that a peptide mimic may serve equally well as a peptide for the purpose of providing the specific backbone conformation and side chain functionalities required for binding to C3 and inhibiting complement activation. Accordingly, it is contemplated as being within the scope of the present invention to produce C3-binding, complement-inhibiting compounds through the use of either naturally-occurring amino acids, amino acid derivatives, analogs or non-amino acid molecules capable of being joined to form the appropriate backbone conformation. A non-peptide analog, or an analog comprising peptide and non-peptide components, is sometimes referred to herein as a “peptidomimetic” or “isosteric mimetic,” to designate substitutions or derivations of the peptides of the invention, which possess the same backbone conformational features and/or other functionalities, so as to be sufficiently similar to the exemplified peptides to inhibit complement activation.

The use of peptidomimetics for the development of high-affinity peptide analogs is well known in the art (see, e.g., Vagner et al., 2008, Curr. Opin. Chem. Biol. 12: 292-296; Robinson et al., 2008, Drug Disc. Today 13: 944-951) Assuming rotational constraints similar to those of amino acid residues within a peptide, analogs comprising non-amino acid moieties may be analyzed, and their conformational motifs verified, by any variety of computational techniques that are well known in the art.

The modified compstatin peptides of the present invention can be modified by the addition of polyethylene glycol (PEG) components to the peptide. As is well known in the art, PEGylation can increase the half-life of therapeutic peptides and proteins in vivo. In one embodiment, the PEG has an average molecular weight of about 1,000 to about 50,000. In another embodiment, the PEG has an average molecular weight of about 1,000 to about 20,000. In another embodiment, the PEG has an average molecular weight of about 1,000 to about 10,000. In an exemplary embodiment, the PEG has an average molecular weight of about 5,000. The polyethylene glycol may be a branched or straight chain, and preferably is a straight chain.

The compstatin analogs of the present invention can be covalently bonded to PEG via a linking group. Such methods are well known in the art. (Reviewed in Kozlowski A. et al. 2001, BioDrugs 15: 419-29; see also, Harris J M and Zalipsky S, eds. Poly(ethylene glycol), Chemistry and Biological Applications, ACS Symposium Series 680 (1997)). Non-limiting examples of acceptable linking groups include an ester group, an amide group, an imide group, a carbamate group, a carboxyl group, a hydroxyl group, a carbohydrate, a succinimide group (including without limitation, succinimidyl succinate (SS), succinimidyl propionate (SPA), succinimidyl carboxymethylate (SCM), succinimidyl succinamide (SSA) and N-hydroxy succinimide (NHS)), an epoxide group, an oxycarbonylimidazole group (including without limitation, carbonyldimidazole (CDI)), a nitro phenyl group (including without limitation, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine. In certain embodiments, the linking group is a succinimide group. In one embodiment, the linking group is NHS.

The compstatin analogs of the present invention can alternatively be coupled directly to PEG (i.e., without a linking group) through an amino group, a sulfhydral group, a hydroxyl group or a carboxyl group. In one embodiment, PEG is coupled to a lysine residue added to the C-terminus of compstatin.

As an alternative to PEGylation, the in vivo clearance of peptides can also be reduced by linking the peptides to certain other molecules or peptides. For instance, certain albumin binding peptides display an unusually long half-life of 2.3 h when injected by intravenous bolus into rabbits (Dennis et al., 2002, J Biol Chem. 277: 35035-35043). A peptide of this type, fused to the anti-tissue factor Fab of D3H44 enabled the Fab to bind albumin while retaining the ability of the Fab to bind tissue factor (Nguyen et al., 2006, Protein Eng Des Sel. 19: 291-297.). This interaction with albumin resulted in significantly reduced in vivo clearance and extended half-life in mice and rabbits, when compared with the wild-type D3H44 Fab, comparable with those seen for PEGylated Fab molecules, immunoadhesins, and albumin fusions. WO2010/127336 sets forth suitable synthesis strategies utilizing an ABP as well as an albumin-binding small molecule (ABM), and optionally employing a spacer between the components. Those procedures resulted in the production of conjugates of ABP- and ABM-compstatin analogs capable of inhibiting complement activation and also exhibiting extended in vivo survival. Indeed, the ABP was able to improve the half-life of a compstatin analog by 21 fold without significantly compromising its inhibitory activity. Thus, such conjugates enable the systemic administration of the inhibitor without infusion.

The binding properties and complement activation-inhibiting activity of compstatin analogs, peptidomimetics and conjugates may be tested by a variety of assays known in the art. In various embodiments, the assays described in Example 1 are utilized. A non-exhaustive list of other assays is set forth in U.S. Pat. No. 6,319,897, WO99/13899, WO2004/026328 and WO2007/062249, including, but not limited to, (1) peptide binding to C3 and C3 fragments; (2) various hemolytic assays; (3) measurement of C3 convertase-mediated cleavage of C3; and (4) measurement of Factor B cleavage by Factor D.

The peptides and peptidomimetics described herein are of practical utility for any purpose for which compstatin itself is utilized, as known in the art. Such uses include, but are not limited to: (1) inhibiting complement activation in the serum, tissues or organs of a patient (human or animal), which can facilitate treatment of certain diseases or conditions, including but not limited to, age-related macular degeneration, rheumatoid arthritis, spinal cord injury, Parkinson's disease, Alzheimer's disease, cancer, and respiratory disorders such as asthma, chronic obstructive pulmonary disease (COPD), allergic inflammation, emphysema, bronchitis, bronchiecstasis, cyctic fibrosis, tuberculosis, pneumonia, respiratory distress syndrome (RDS—neonatal and adult), rhinitis and sinusitis; (2) inhibiting complement activation that occurs during cell or organ transplantation, or in the use of artificial organs or implants (e.g., by coating or otherwise treating the cells, organs, artificial organs or implants with a peptide of the invention); (3) inhibiting complement activation that occurs during extracorporeal shunting of physiological fluids (blood, urine) (e.g., by coating the tubing through which the fluids are shunted with a peptide of the invention); and (4) in screening of small molecule libraries to identify other inhibitors of compstatin activation (e.g., liquid- or solid-phase high-throughput assays designed to measure the ability of a test compound to compete with a compstatin analog for binding with C3 or a C3 fragment).

To implement one or more of the utilities mentioned above, another aspect of the invention features pharmaceutical compositions comprising the compstatin analogs or conjugates described and exemplified herein. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The formulations of the pharmaceutical compositions may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-does unit.

As used herein, the term “pharmaceutically-acceptable carrier” means a chemical composition with which a complement inhibitor may be combined and which, following the combination, can be used to administer the complement inhibitor to a mammal.

The following example is provided to describe the invention in greater detail. It is intended to illustrate, not to limit, the invention.

### Example 1

Cystathionine compstatin analogs were synthesized in accordance with standard methods and the schemes set forth below. Schematic representations of the analogs and the orientation of their respective thioether bonds are shown in FIG. 1.

A. Synthesis of Monomers:

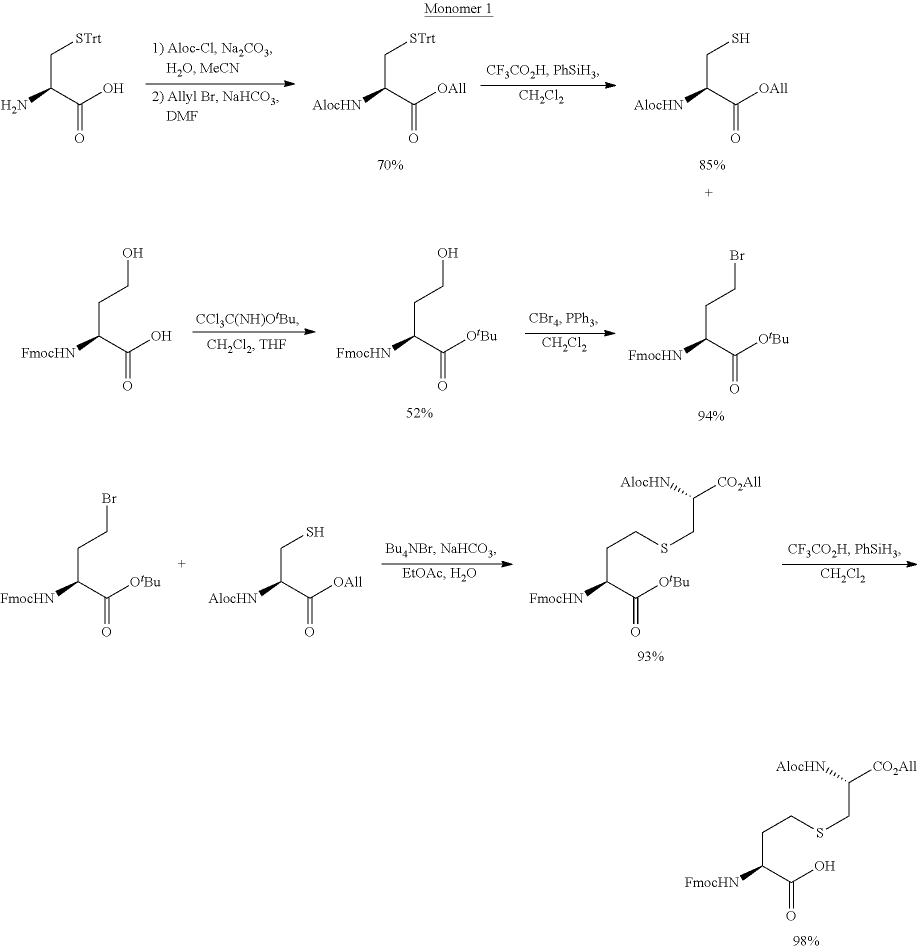

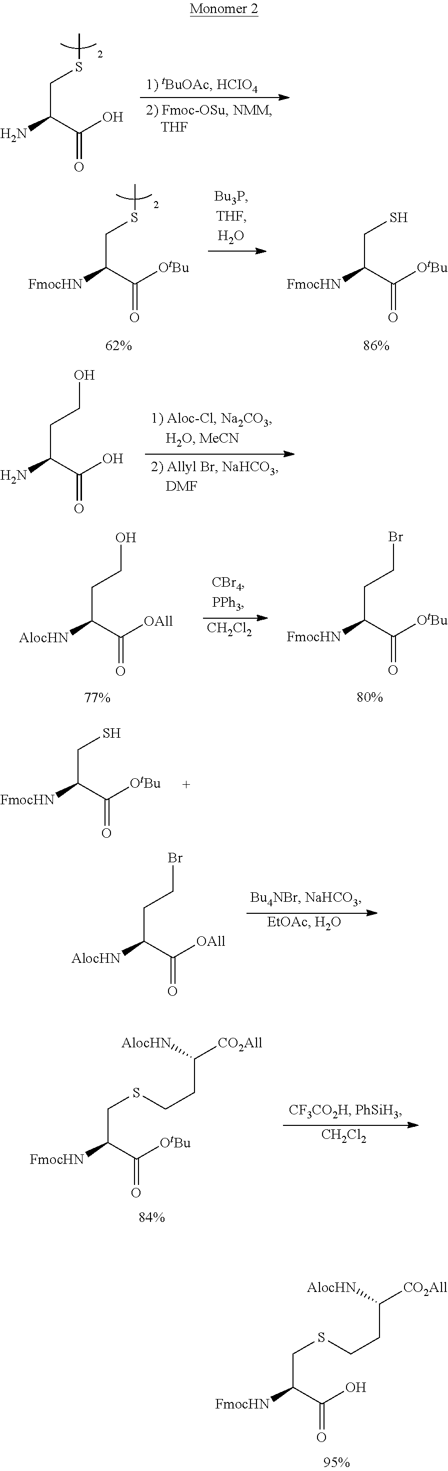

B. Synthesis of W49A Cystathionine Compstatin Analogs (the CP20 Compstatin Analog was Sythesized Similarly):

### Gamma-Cystathionine Analog

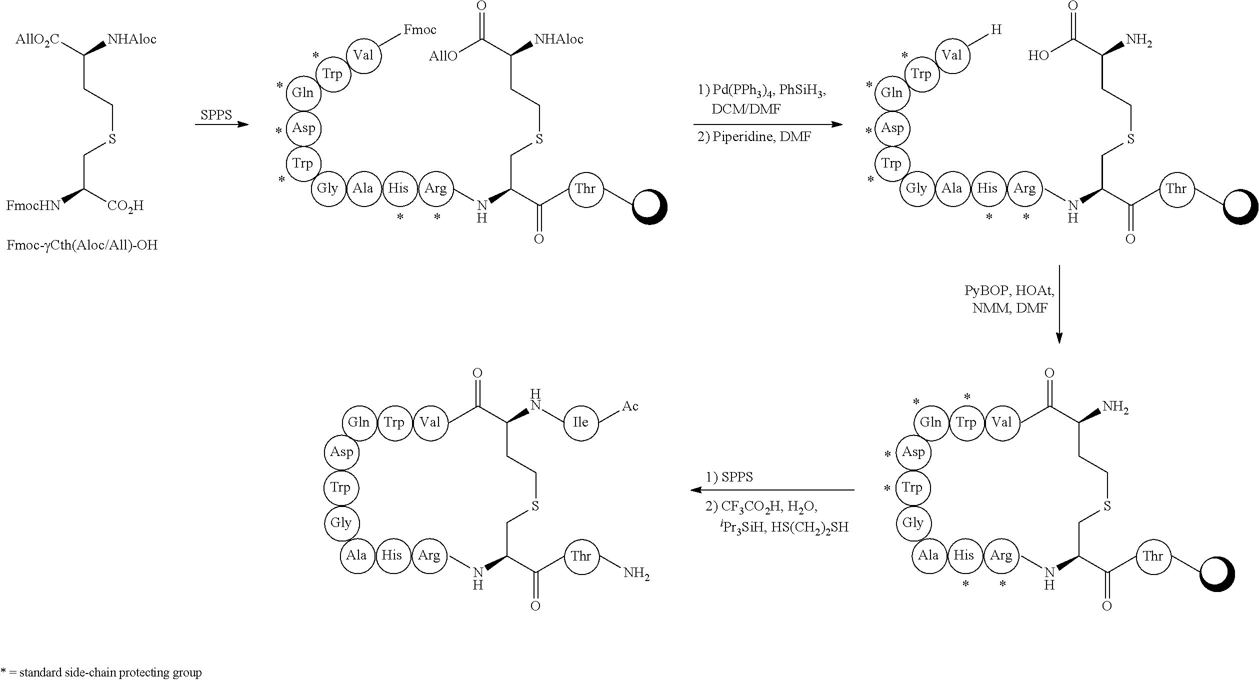

### Delta-Cystathionine Analog

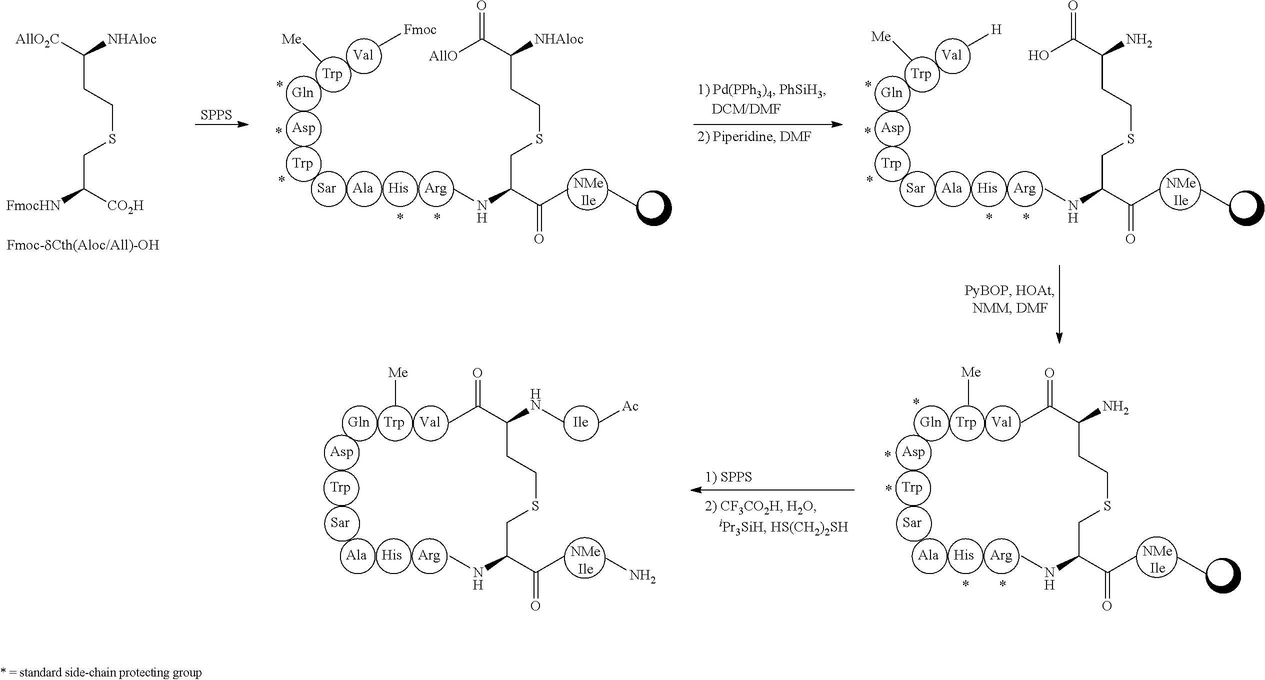

Complement Inhibition Analysis.

An ELISA-based assay was performed to assess the complement inhibitory ability of the compstatin analogs. Briefly, an antigen-antibody complex was used as an initiator of the classical pathway of complement activation. Serial dilutions of the compstatin analogs were prepared and plasma was added for a final dilution of 1:80 in VB++ (Veronal Buffer saline containing MgCl₂ and CaCl₂). Incubation followed to allow complement activation. The deposition of C3b on the plate was measured by a polyclonal anti-C3 antibody. The percentage of inhibition was plotted against the compstatin concentration and fitted to a logistic dose-response function using OriginPro 8.

Kinetic Analysis.

The interaction of the compstatin analogs with C3b was characterized using a Biacore 3000 instrument (GE Healthcare, Corp., Piscataway, N.J.). The running buffer was PBS, pH 7.4 (10 mM sodium phosphate, 150 mM NaCl) with 0.005% Tween-20. Biotinylated C3b was captured site-specifically on a streptravidin chip at about 3000, 4000 and 5000 RU density; an untreated flowcell was used as a reference surface. For kinetic analysis, sets of five samples of increasing concentrations were injected over the chip surface one after the other in a single cycle. Three-fold dilution series (0.46-37 nM for delta-Cth CP20 and CP20 and 0.46-37 nM and 111-9000 nM for the other analogs) were injected at 30 ul/min; each injection was done for 2 min, allowing every time the peptide to dissociate for 5 min before the next injection started. The data analysis was performed using BiaEvaluation. The processed signals were fitted to a 1:1 binding model and kinetic constants were extracted. The analogs gamma-cystathionine (gamma-Cth), delta-cystathionine (delta-Cth), and 4W9A (control) were tested both with the above assay as well as with the following method: Different concentrations were injected over the chip surface in different cycles. 2 min injections at 30 ul/min were performed, after which a 3 min dissociation was monitored. A three-fold dilution series (1.8 nM-36 uM) was used. Data analysis followed using Scrubber (BioLogic Software, Campbell, Australia). The processed signals were globally fitted to a Langmuir 1:1 binding isotherm and kinetic constants were extracted.

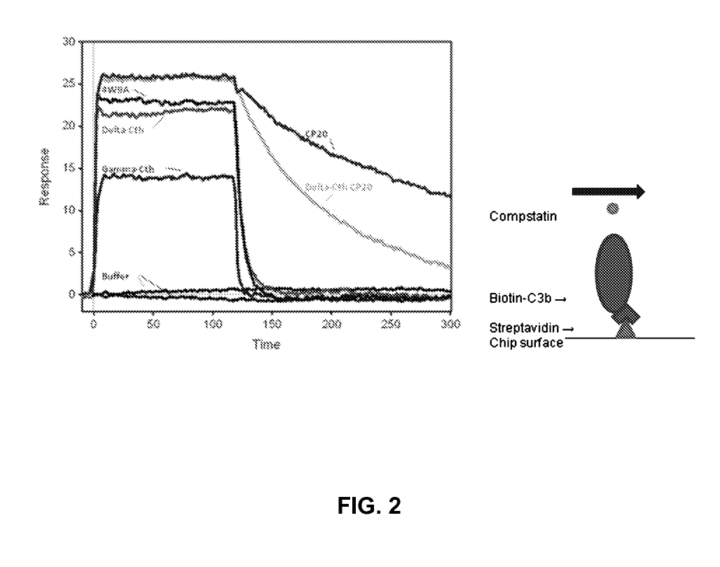

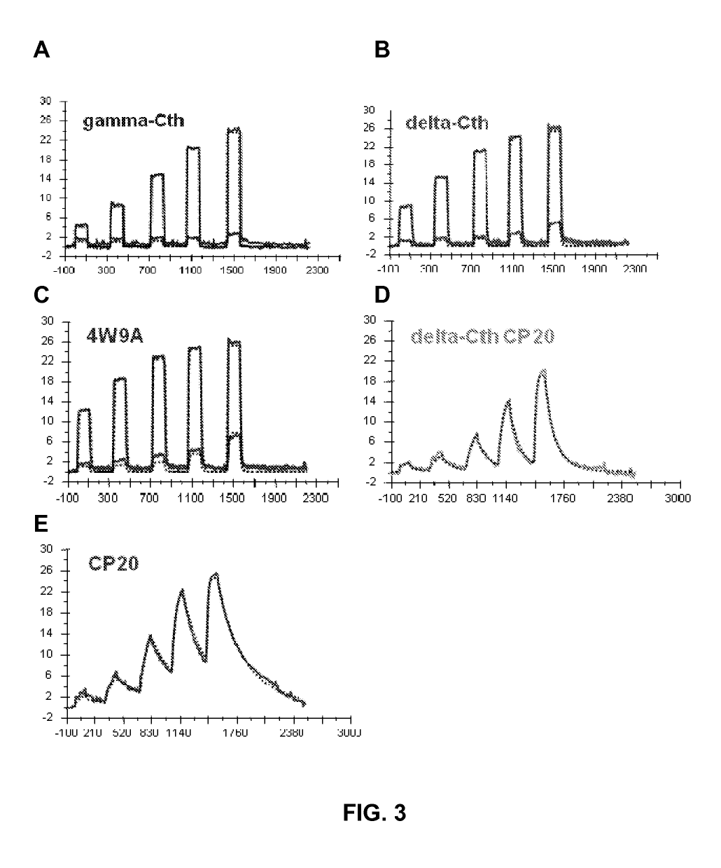

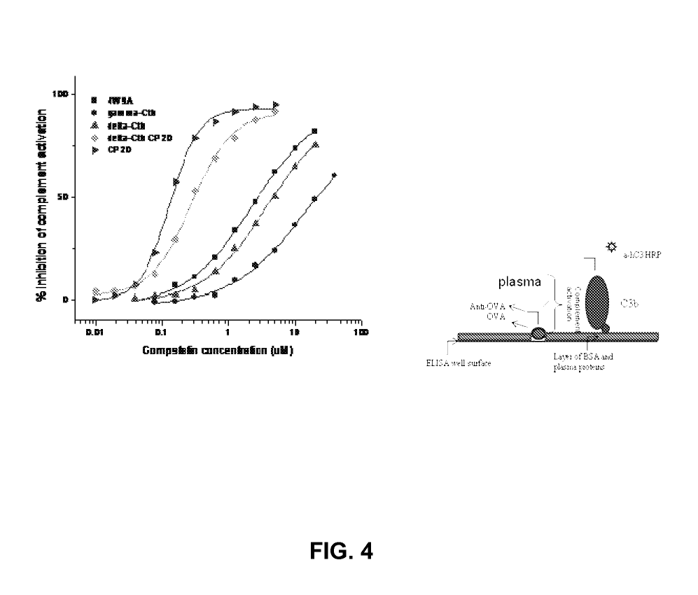

Results are shown in FIGS. 2-4 and in Table 1 and Table 2 below.

TABLE 1
Kinetic analysis of compstatin analogs - average of multiple datasets.

| | kₐ (10⁵ M⁻¹s⁻¹) | k_{d} (10⁻² s⁻¹) | K_{D} (nM) |
|---|---|---|---|
| Gamma-Cth | 5.5 ± 0.7 | 46.8 ± 5.7 | 847 ± 59 |
| Delta-Cth | 4.6 ± 0.5 | 11 ± 1.9 | 236 ± 20 |
| Delta-Cth CP20 | 16.4 ± 3.8 | 1.5 ± 0.4 | 9 ± 1.8 |
| 4W9A | 5.6 ± 1.4 | 8.1 ± 1.1 | 150 ± 19 |
| CP20 | 18.6 ± 4.1 | 0.4 ± 0.1 | 2.4 ± 0.6 |

TABLE 2
Concentration at which the peptides inhibit complement activation
by 50% (IC50) for the most active analogs - average of five datasets.

| | IC₅₀ (uM) |
|---|---|
| Delta-Cth | 3.3 ± 2 |
| Delta-Cth CP20 | 0.26 ± 0.09 |
| 4W9A | 1.9 ± 1.3 |
| CP20 | 0.13 ± 0.05 |

The present invention is not limited to the embodiments described and exemplified above, but is capable of variation and modification within the scope of the appended claims.

1. A compound comprising a modified compstatin peptide (ICVVQDWGHHRCT (disulfide C2-C12; SEQ ID NO:1) or analog thereof, in which the disulfide bond between C2 and C12 is replaced with a thioether bond to form a cystathionine.

2. The compound of claim 1, wherein the cystathionine is a delta-cystathionine.

3. The compound of claim 2, further comprising replacement of His at position 9 with Ala.

4. The compound of claim 3, further comprising replacement of Val at position 4 with Trp or an analog of Trp.

5. The compound of claim 4, wherein the analog of Trp at position 4 is 1-methyl Trp or 1-formyl Trp.

6. The compound of claim 4, further comprising replacement of Trp at position 7 with an analog of Trp.

7. The compound of claim 6, wherein the analog of Trp at position 7 is a halogenated Trp.

8. The compound of claim 3, further comprising acetylation of the N-terminal residue.

9. The compound of claim 1, further comprising modification of Gly at position 8 to constrain the backbone conformation at that location.

10. The compound of claim 9, wherein the backbone is constrained by replacing the Gly at position 8 (Gly8) with Nα-methyl Gly.

11. The compound of claim 9, further comprising replacing the Thr at position 13 with Ile, Leu, Nle, N-methyl Thr or N-methyl Ile.

12. The compound of claim 1, which is a compstatin analog comprising a peptide having a sequence of SEQ ID NO:2, which is:
Xaa1-Cys-Val-Xaa2-Gln-Asp-Xaa3-Gly-Xaa4-His-Arg-
Cys-Xaa5
(cystathionine C2-C12) in which Gly at position 8 is modified to constrain the backbone conformation at that location;
wherein:
Xaa1 is Ile, Val, Leu, Ac-Ile, Ac-Val, Ac-Leu or a dipeptide comprising Gly-Ile;
Xaa2 is Trp or an analog of Trp, wherein the analog of Trp has increased hydrophobic character as compared with Trp;
Xaa3 is Trp or an analog of Trp comprising a chemical modification to its indole ring wherein the chemical modification increases the hydrogen bond potential of the indole ring;
Xaa4 is His, Ala, Phe or Trp; and
Xaa5 is Thr, Ile, Leu, Nle, N-methyl Thr or N-methyl Ile, wherein a carboxy terminal —OH of any of the Thr, Ile, Leu, Nle, N-methyl Thr or N-methyl Ile optionally is replaced by —NH₂.

13. The compound of claim 12, wherein:
the cystathionine is a delta-cystathionine;
the Gly at position 8 is N-methylated;
Xaa1 is Ac-Ile;
Xaa2 is Trp, 1-methyl-Trp or 1-formyl-Trp;
Xaa3 is Trp;
Xaa4 is Ala; and
Xaa5 is Thr, Ile, Leu, Nle, N-methyl Thr or N-methyl Ile.

14. The compound of claim 13, wherein Xaa5 is Ile, N-methyl Thr or N-methyl Ile.

15. The compound of claim 13, which comprises SEQ ID NO:5 or SEQ ID NO:7.

16. The compound of claim 1, further comprising an additional component that extends the in vivo retention of the compound.

17. The compound of claim 16, wherein the additional component is polyethylene glycol (PEG).

18. The compound of claim 16, wherein the additional component is an albumin binding small molecule.

19. The compound of claim 16, wherein the additional component is an albumin binding peptide.

20. The compound of claim 19, wherein the albumin binding peptide comprises the sequence RLIEDICLPRWGCLWEDD (SEQ ID NO: 8).

21. The compound of claim 19, wherein the compound and the albumin binding peptide are separated by a spacer.

22. The compound of claim 21, wherein the spacer is a polyethylene glycol molecule.

23. A pharmaceutical composition comprising a modified compstatin peptide (ICVVQDWGHHRCT (disulfide C2-C12; SEQ ID NO:1) or analog thereof, in which the disulfide bond between C2 and C12 is replaced with a thioether bond to form a cystathionine, and a pharmaceutically acceptable carrier.

24. (canceled)

25. A compound that inhibits complement activation, comprising a non-peptide or partial peptide mimetic of SEQ ID NO:5 or SEQ ID NO:7, wherein the compound binds C3 and inhibits complement activation with at least 500-fold greater activity than does a peptide comprising SEQ ID NO:1 under equivalent assay conditions.


SEQUENCE LISTING (8 sequences)

SEQ ID NO: 1 - protein, 13 aa, Artificial Sequence
  Compstatin
ICVVQDWGHHRCT

SEQ ID NO: 2 - protein, 14 aa, Artificial Sequence
  Compstatin analog
XXCVXQDXGXHRCX

SEQ ID NO: 3 - protein, 13 aa, Artificial Sequence
  Compstatin analog 4W9A
ICVWQDWGAHRCT

SEQ ID NO: 4 - protein, 13 aa, Artificial Sequence
  Thioether compstatin analog 4W9A gamma-Cth
ICVWQDWGAHRCT

SEQ ID NO: 5 - protein, 13 aa, Artificial Sequence
  Thioether compstatin analog 4W9A delta-Cth
ICVWQDWGAHRCT

SEQ ID NO: 6 - protein, 13 aa, Artificial Sequence
  Compstatin analog CP20
ICVWQDWGAHRCI

SEQ ID NO: 7 - protein, 13 aa, Artificial Sequence
  Thioether compstatin analog CP20 delta-Cth
ICVWQDWGAHRCI

SEQ ID NO: 8 - protein, 18 aa, Artificial Sequence
  Albumin binding peptide
RLIEDICLPRWGCLWEDD